(19) United States
(12) Patent Application Publication
(10) Pub. No.: US 2013/0035653 A1
(43) Pub. Date: Feb. 7, 2013

(54) Ostomy Pouch Comprising Absorbent Material

(71) Applicants: Charles Kannankeril, North Caldwell, NJ (US); Rick Merical, Duncan, SC (US)

(72) Inventors: Charles Kannankeril, North Caldwell, NJ (US); Rick Merical, Duncan, SC (US)

(73) Assignee: CRYOVAC, INC., Duncan, SC (US)

(21) Appl. No.: 13/196,361

(22) Filed: Aug. 2, 2011

(51) Int. Cl. A61F 5/441; B31B 49/00; A61F 5/445

(52) U.S. Cl. 604/333; 604/332; 493/267

(57) ABSTRACT

The presently disclosed subject matter is directed to an ostomy pouch comprising at least one absorbent material housed within the interior of the pouch. Specifically, the absorbent materials are contained within a sachet that includes at least one of the following: a frangible seal, a porous film, or a fluid-permeable film. In use, as fluid enters the interior of the ostomy pouch and contacts the sachet, the porous film and/or the frangible seal weakens to allow fluid to enter the sachet and contact the absorbent material housed within. Alternatively or in addition, in embodiments wherein the sachet includes a fluid-permeable film, fluid directly enters the interior of the sachet. As a result, the absorbent material expands and ruptures the frangible seal and/or the sachet, thereby releasing the absorbent material into the interior of the pouch for full absorption of the fluid.

### FIELD OF THE INVENTION

The presently disclosed subject matter relates generally to ostomy pouches comprising one or more absorbent materials, such as superabsorbent polymers. Particularly, the absorbent material is secured within the ostomy pouch by means of a sachet comprising at least one frangible seal.

### BACKGROUND

Treating some diseases of the digestive or urinary systems can involve removing all or part of a patient's small intestine, colon, rectum or bladder. In these cases, wastes must be rerouted to exit the body of the subject. The rerouting surgery, known as an ostomy, involves diverting a biological system (e.g., colon, ileum, urinary) to a newly created opening in the abdomen wall called a stoma. In an ileostomy, the colon and rectum are surgically removed and the bottom of the small intestine (ileum) is connected to a stoma. In a colostomy, the surgeon removes the rectum and attaches the colon to a stoma. In a urostomy, the ureters are rerouted to either the small intestine or to the abdominal wall.

An ostomy pouching system (colloquially called a colostomy bag) is a medical prosthetic that provides a means for the collection of waste from a diverted biological system (colon, ileum, urinary) as it exists the stoma. Pouching systems usually consist of a mounting plate, commonly called a wafer, and a collection pouch that is mechanically attached in an airtight seal. The pouching system allows the stoma to drain into a sealed collection pouch, while protecting the surrounding skin from contamination. Ostomy pouching systems are air-tight and fluid-tight and allow the wearer to lead an active lifestyle. Pouching systems come in one-piece and two-piece designs. In the one-piece design, the ostomy pouch interacts directly with the stoma and surrounding area, forming a seal over the stoma. In the two-piece design, a wafer is attached to the skin, often with a “peel and stick” adhesive backing, making a fluid tight seal around the stoma, and providing a reusable attachment site for the ostomy pouch. The selection of systems varies greatly between individuals and is often based on personal preference and lifestyle.

Traditional ostomies generally require the user to have a pouch or other container attached to the ostomy for constant collection of body waste. Necessarily, the pouch will become heavy and cumbersome as it automatically fills with body waste over time, and the user is faced with the risk of spillage during use as well as during the process of emptying the pouch contents. In addition, as they fill with wastes, traditional ostomy pouches can make noise during user movement, resulting increased self-consciousness. In response, prior art ostomy pouches conventionally include absorbent materials deposited in the bottom of the pouch. However, placing such loose absorbent materials within the pouch is difficult to control. In addition, the loose absorbent materials can move around and contaminate the pouch valve and spout area. Alternatively, an absorbent material sachet can be introduced into the pouch by the user through a pouch opening. However, the use of such absorbent sachets in this manner provides the temptation for a patient to reuse the ostomy pouch (i.e., insert another absorbent sachet into the pouch). For safety reasons, reusing ostomy pouches is not desirable. Further, the addition of the absorbent sachet into the interior of the ostomy pouch by the patient can impose contamination into the interior of the pouch. Moreover, the user must carry a separate product (the absorbent sachet) and must ensure that the sachet does not rupture when introducing it through the narrow spout into the interior of the ostomy pouch.

To combat the negative attributes associated with the prior art ostomy pouches described above, the presently disclosed subject matter comprises an ostomy pouch that includes one or more absorbent materials (such as superabsorbent polymers) immobilized within a sachet comprising at least one frangible seal. Specifically, the sachet secures the absorbent materials, while allowing fluids to pass through at least one of the sachet films and/or the frangible seal. As the sachet contacts fluid, at least one sachet film and/or frangible seal is weakened, thereby allowing the fluid to enter the interior of the sachet. Alternatively, in embodiments wherein the sachet is constructed from a fluid-permeable film, fluid passes directly into the interior of the sachet. When the absorbent material contacts and absorbs the fluid, it expands and the volume inside the sachet increases. The resultant increase in pressure ruptures the sachet and/or frangible seal, thereby freeing the full amount of absorbent material into the interior of the pouch for full absorption of the fluid.

Other features, advantages and objects of the invention will become apparent from the following description.

### SUMMARY

In some embodiments, the presently disclosed subject matter is directed to an ostomy pouch comprising a first wall and a second wall joined together to define a closed compartment having an interior. The ostomy pouch also comprises an aperture formed in one of the first or second walls of the pouch and in communication with the pouch interior. Further, the ostomy pouch comprises a sachet positioned within the pouch interior. The sachet comprises at least one of the following: a frangible seal, a soluble film, or a fluid-permeable film. The sachet also comprises at least one absorbent material housed within the interior of the sachet. The sachet is configured such that the frangible seal, soluble film, or fluid-permeable film allows fluid to contact the absorbent material housed within the interior of the sachet, thereby increasing the pressure within the sachet interior and rupturing the frangible seal, soluble film, fluid-permeable film, or combinations thereof.

In some embodiments, the presently disclosed subject matter is directed to a method of making an ostomy pouch. The method comprises forming a pouch comprising a first wall and a second wall joined together to define a closed compartment having an interior. The method further comprises providing an aperture formed in one of the first or second walls of the pouch and in communication with the pouch interior. Further, the method comprises providing a sachet positioned within the pouch interior, the sachet comprising at least one of the following: a frangible seal, a soluble film, or a fluid permeable film. The sachet also comprises at least one absorbent material housed within the interior of the sachet. The sachet is configured such that the frangible seal, soluble film, or fluid-permeable film allows fluid to contact the absorbent material housed within the interior of the sachet, thereby increasing the pressure within the sachet interior and rupturing the frangible seal, soluble film, fluid-permeable film, or combinations thereof.

In some embodiments, the presently disclosed subject matter is directed to a method of using an ostomy pouch, the method comprising providing a pouch comprising a first wall and a second wall joined together to define a closed compartment having an interior. The pouch also comprises an aperture formed in one of the first or second walls of the pouch and in communication with the pouch interior. The pouch further comprises a sachet positioned within the pouch interior, the sachet comprising at least one of the following: a frangible seal, a soluble film, or a fluid-permeable film. The sachet also comprises at least one absorbent material housed within the interior of the sachet. The method further comprises attaching the aperture to the stomal area of a user. The sachet is configured such that the frangible seal, soluble film, or fluid-permeable film allows fluid to contact the absorbent material housed within the interior of the sachet, thereby increasing the pressure within the sachet interior and rupturing the frangible seal, soluble film, fluid-permeable film, or combinations thereof.

### BRIEF DESCRIPTION OF THE DRAWINGS

FIG. 1a is a top plan view of an ostomy pouch according to some embodiments of the presently disclosed subject matter.

FIG. 1b is a top plan view of the ostomy pouch of FIG. 1a during or after use.

FIG. 2a is a top plan view of an ostomy pouch according to some embodiments of the presently disclosed subject matter.

FIG. 2b is a side elevation view of the pouch of FIG. 2a.

FIG. 3 is a side elevation view of the pouch of FIG. 2a, further comprising a cover in accordance with some embodiments of the presently disclosed subject matter.

FIG. 4a is top plan view of one embodiment of a sachet that can be used in accordance with the presently disclosed subject matter.

FIG. 4b is a side elevation view of the sachet of FIG. 4a, taken along line 4b -4b.

FIG. 4c is a top elevation view of a sachet that can be used with some embodiments of the presently disclosed subject matter.

FIG. 5a is a top plan view of an ostomy pouch according to some embodiments of the presently disclosed subject matter.

FIG. 5b is a side elevation view of the ostomy pouch of FIG. 5a.

FIG. 5c is an enlarged fragmentary sectional view of one embodiment of a sachet that can be used with the presently disclosed subject matter.

FIG. 5d is a top plan view of one embodiment of an ostomy pouch in accordance with the presently disclosed subject matter.

FIG. 5e is a side elevation view of the pouch of FIG. 5d.

FIGS. 6a and 6b are side elevation views that illustrate one embodiment of the presently disclosed ostomy pouch in use.

FIG. 7a is a top plan view of one embodiment of the presently disclosed sachet during use.

FIGS. 7b-7e are side elevation views of sachets of the presently disclosed subject matter during use in some embodiments.

### DETAILED DESCRIPTION

### I. General Considerations

The presently disclosed subject matter is directed to an ostomy pouch comprising at least one absorbent material (such as, for example, a superabsorbent polymer) housed within the interior of the pouch. Specifically, the absorbent materials are contained within a sachet that includes at least one of the following: a frangible seal, a porous film, or a fluid-permeable film. In use, as fluid enters the interior of the ostomy pouch and contacts the sachet, the porous film and/or the frangible seal weakens to allow fluid to enter the sachet and contact the absorbent material housed within. Alternatively or in addition, in embodiments wherein the sachet includes a fluid-permeable film, fluid directly enters the interior of the sachet. As a result, the absorbent material expands and ruptures the frangible seal and/or the sachet, thereby releasing the absorbent material into the interior of the pouch for full absorption of the fluid.

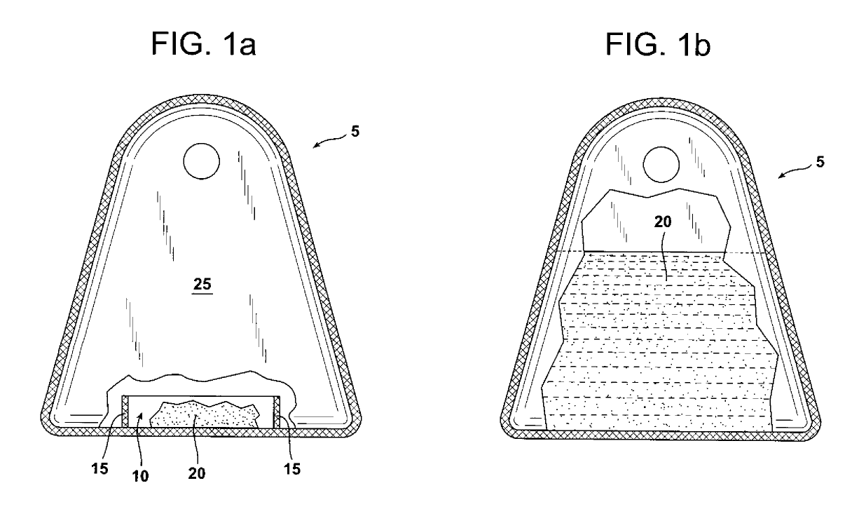

FIG. 1a illustrates one embodiment of an ostomy pouch in accordance with the presently disclosed subject matter. Specifically, FIG. 1a illustrates ostomy pouch 5 comprising sachet 10 disposed within the interior of the pouch. Sachet 10 includes at least one frangible seal 15 and houses absorbent material 20. FIG. 1b illustrates the ostomy pouch of FIG. 1a after contact with bodily fluids has breached a frangible seal and/or film of sachet 10, thereby rupturing the sachet film and/or frangible seal 15 and fully expanding the absorbent material. Methods of making and using pouch 5 are also included within the scope of the presently disclosed subject matter.

### II. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long standing patent law convention, the terms “a”, “an”, and “the” refer to “one or more” when used in the subject application, including the claims. Thus, for example, reference to “a pouch” includes a plurality of such pouches, and so forth.

Unless indicated otherwise, all numbers expressing quantities of components, reaction conditions, and so forth used in the specification and claims are to be understood as being modified in all instances by the term “about.” Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, percentage, and the like can encompass variations of, and in some embodiments, ±20%, in some embodiments ±10%, in some embodiments ±5%, in some embodiments ±1%, in some embodiments ±0.5%, and in some embodiments ±0.1%, from the specified amount, as such variations are appropriated in the disclosed package and methods.

The term “absorbent material” as used herein refers to materials that receive and contain fluids, such as bodily exudates. Thus, in some embodiments, an absorbent material in accordance with the presently disclosed subject matter can imbibe and hold fluids.

As used herein, the phrase “abuse layer” refers to an outer film layer and/or an inner film layer, so long as the film layer serves to resist abrasion, puncture, and other potential causes of reduction of package integrity, as well as potential causes of reduction of package appearance quality. Abuse layers can comprise any polymer, so long as the polymer contributes to achieving an integrity goal and/or an appearance goal; preferably, abuse layers comprise polymer having a modulus of at least 107 Pascals, at room temperature; more preferably, the abuse layer comprises at least one member selected from the group consisting of polyolefin (ethylene vinyl acetate, low density polyethylene, linear low density polyethylene, metallocene linear low density polyethylene, and the like), polyamide, ethylene/propylene copolymer; more preferably, nylon 6, nylon 6/6, amorphous nylon, and ethylene/propylene copolymer.

As used herein, the term “aperture” refers to an opening or open space (such as a hole, gap, cleft, or slit) in a film or material.

The term “bactericide” refers to any of a wide variety of materials capable of reducing the rate of bacterial growth. Suitable bactericides can include (but are not limited to) halogen compounds, iodide salts, chloramine, chlorohexidine, sodium hypochlorite, silver and silver-containing compounds, zinc and zinc salts, oxidants (such as hydrogen peroxide and potassium permanganate), aryl mercury compounds, alkyl mercury compounds, phenols, activated charcoal, and organic nitrogen compounds.

As used herein, the terms “barrier” and “barrier layer” as applied to films and/or film layers, refer to the ability of a film or film layer to serve as a barrier to gases and/or odors. Examples of polymeric materials with low oxygen transmission rates useful in such a layer can include: ethylene/vinyl alcohol copolymer (EVOH), polyvinylidene dichloride (PVDC), vinylidene chloride copolymer such as vinylidene chloride/methyl acrylate copolymer, vinylidene chloride/vinyl chloride copolymer, polyamide, polyglycolic acid, polyester, polyacrylonitrile (available as Barex™ Resin), or blends thereof. Oxygen barrier materials can further comprise high aspect ratio fillers that create a tortuous path for permeation (e.g., nanocomposites). Oxygen barrier properties can be further enhanced by the incorporation of an oxygen scavenger, such as an organic oxygen scavenger. In some embodiments, metal foil, metallized substrates (e.g., metallized polyethylene terephthalate ((PET)), metallized polyamide, and/or metallized polypropylene), and/or coatings comprising SiOx or AlOx compounds can be used to provide low oxygen transmission to a package. In some embodiments, a barrier layer can have a gas (e.g., oxygen) permeability of less than or equal to about 500 cc/m²/24 hrs/atm at 73° F., in some embodiments less than about 100 cc/m²/24 hrs/atm at 73° F., in some embodiments less than about 50 cc/m²/24 hrs/atm at 73° F., and in some embodiments less than about 25 cc/m²/24 hrs/atm at 73° F.

The term “bulk layer” as used herein refers to a layer used to increase the abuse-resistance, toughness, modulus, etc., of a film. In some embodiments, the bulk layer can comprise polyolefin (including but not limited to) at least one member selected from the group comprising ethylene/alpha-olefin copolymer, ethylene/alpha-olefin copolymer plastomer, low density polyethylene, and/or linear low density polyethylene and polyethylene vinyl acetate copolymers.

The term “disposable” as used herein describes devices (such as ostomy pouches) that are generally not intended to be laundered or otherwise restored or reused (i.e., they are intended to be discarded after a single use).

As used herein, the term “film” can be used in a generic sense to include plastic web, regardless of whether it is film or sheet. In some embodiments, the term “film” can include a nonwoven fabric, paper tissue, and like materials.

The term “fluid” as used herein refers to liquids, gases, semi-solids, pastes, gels, and combinations thereof. In some embodiments, the term “fluid” can include any substance that is not a solid.

The term “fluid permeable” as used herein refers to a material that is capable of allowing fluid (such as, but not limited to, urine) present on one side of the material to get to the opposite side of the material, irrespective of the way and/or the mechanism through which this is accomplished.

As used herein, the term “frangible seal” refers to a seal that is sufficiently durable to allow normal handling thereof, yet that can peel or substantially separate in response to a condition, such as exposure to a fluid.

As used herein, the term “polymer” refers to the product of a polymerization reaction, and can be inclusive of homopolymers, copolymers, terpolymers, etc. In some embodiments, the layers of a film can consist essentially of a single polymer, or can have additional polymer together therewith, i.e., blended therewith.

The term “pouch” as used herein is not limiting and includes the wide variety of containers known in the art, including (but not limited to) bags, packets, packages, and the like. In some embodiments, the term “pouch” includes any protective or collective device having an opening adapted to be secured about a stoma for protecting the user and for collecting exudate. Such pouches are well known in the art; e.g., U.S. Pat. Nos. 3,827,435; 3,954,105; 4,205,678; 4,268,286; 4,983,171; and 5,074,851, the entire disclosures of which are hereby incorporated by reference herein.

The term “sachet” as used herein refers to a closed receptacle for housing one or more absorbent materials. The sachet is closed in the sense that the absorbent materials, prior to initiation, are substantially retained within the sachet. The term “sachet” is not intended to be limiting and can include any of a wide variety of receptacles known in the art, including (but not limited to) pouches, bags, envelopes, capsules, packets, and containers. In some embodiments, the term “sachet” can include a receptacle formed from one wall or area of a pouch and a portion of film.

As used herein, the term “seal” refers to any seal of a first region of an outer film surface to a second region of an outer film surface, including heat or any type of adhesive material, thermal or otherwise. In some embodiments, the seal can be formed by heating the regions to at least their respective seal initiation temperatures. The sealing can be performed by any one or more of a wide variety of means, including (but not limited to) using a heat seal technique (e.g., melt-bead sealing, thermal sealing, impulse sealing, dielectric sealing, radio frequency sealing, ultrasonic sealing, hot air, hot wire, infrared radiation, pressure sensitive adhesives, UV curing adhesive, and the like).

As used herein, the phrases “seal layer”, “sealing layer”, “heat seal layer”, and “sealant layer”, refer to an outer film layer, or layers, involved in the sealing of the film to itself, another film layer of the same or another film, and/or another article that is not a film. It should also be recognized that in general, up to the outer 3 mils of a film can be involved in the sealing of the film to itself or another layer. In general, a sealant layer sealed by heat-sealing layer comprises any thermoplastic polymer. In some embodiments, the heat-sealing layer can comprise, for example, thermoplastic polyolefin, thermoplastic polyamide, thermoplastic polyester, and thermoplastic polyvinyl chloride. In some embodiments, the heat-sealing layer can comprise thermoplastic polyolefin.

The term “soluble film” refers to a film that dissolves in a solvent (such as, for example, fluid). In some embodiments, soluble films suitable for use with the presently disclosed subject matter can include (but are not limited to) films comprising paper, polyvinyl alcohol, or combinations thereof.

As used herein, the term “stoma” refers to the surgically created aperture in the skin and the attached end of the bladder, conduit, or intestine. The stoma provides an open conduit through which a constant or intermittent efflux of waste material occurs. In some embodiments, the term “stoma” can refer to stomas emanating from internal reservoir structures as well as normal, intact bowel.

The term “superabsorbent polymer” (or “SAP”) refers to materials that are capable of absorbing and retaining at least about 10 times their weight in fluids under a 0.5 psi pressure. The superabsorbent polymer particles of the presently disclosed subject matter can be inorganic or organic crosslinked hydrophilic polymers, such as polyvinyl alcohols, polyethylene oxides, crosslinked starches, guar gum, xanthan gum, polyacrylates (i.e., salts of polyacrylic acid), carboxymethyl cellulose, compressed wood pulp fiber, and/or other materials known those in the art of absorbent article manufacture. SAPs are generally discussed in U.S. Pat. Nos. 5,669,894 and 5,599,335, each incorporated herein by reference in their entireties.

As used herein, the term “tie layer” refers to an internal film layer having the primary purpose of adhering two layers to one another. In some embodiments, tie layers can comprise any nonpolar polymer having a polar group grafted thereon, such that the polymer is capable of covalent bonding to polar polymers such as polyamide and ethylene/vinyl alcohol copolymer. In some embodiments, tie layers can comprise at least one member selected from the group including, but not limited to, modified polyolefin, modified ethylene/vinyl acetate copolymer, and/or homogeneous ethylene/alpha-olefin copolymer. In some embodiments, tie layers can comprise at least one member selected from the group consisting of anhydride modified grafted linear low density polyethylene, anhydride grafted low density polyethylene, homogeneous ethylene/alpha-olefin copolymer, and/or anhydride grafted ethylene/vinyl acetate copolymer.

All compositional percentages used herein are presented on a “by weight” basis, unless designated otherwise.

Although the majority of the above definitions are substantially as understood by those of skill in the art, one or more of the above definitions can be defined hereinabove in a manner differing from the meaning as ordinarily understood by those of skill in the art, due to the particular description herein of the presently disclosed subject matter.

### II. Ostomy Pouch 5

II.A. Generally

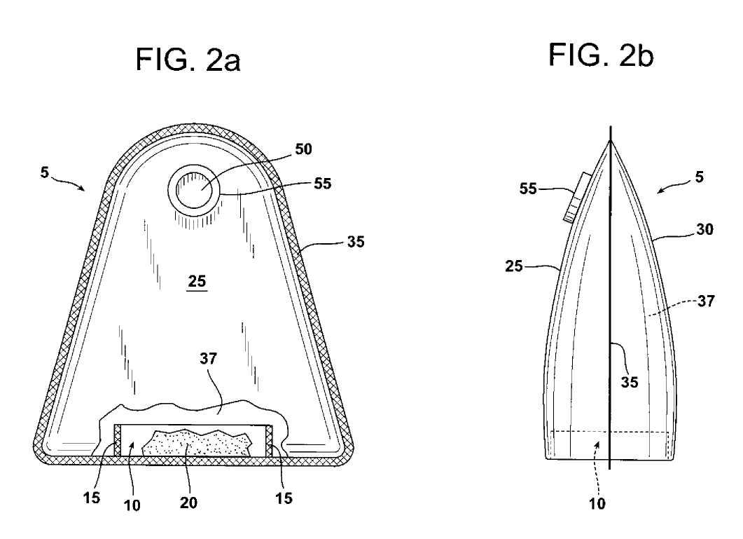

Referring to FIGS. 2a and 2b, pouch 5 includes first wall 25 and second wall 30 of polymeric material. First wall 25 can be considered a rear wall since, when installed, the first wall is positioned adjacent to the user and therefore is not seen when viewing the user. As such, second wall 30 can be considered a front wall since, when installed, the second wall is positioned away from the user and is thus seen. In some embodiments, first and second walls 25 and 30 are welded together around common peripheral seal 35 to define pouch interior 37 for the collection of waste material. It should be appreciated that in lieu of walls 25, 30, the presently disclosed subject matter also includes embodiments wherein pouch 5 is constructed from a unitary piece of material or from a number of separate pieces of material that can be identical or different and that can be sealed or adhered together using means well known in the art. It should also be appreciated that, for the sake of clarity, the drawings show the pouch walls separated from each other (almost inflated) to clearly illustrate the pouch structure. However, during use of the pouch, the pouch will initially be maintained in a flat configuration.

Pouch 5 comprises entrance aperture 50 positioned on one of first and second walls 25, 30, whereby fluid is received prior to storage within the ostomy pouch cavity. In some embodiments, flange 55 is welded to the pouch in register with aperture 50 and forms a mechanical interlock with a complementary coupling member (not shown) worn on the body of the user. Flange 55 can be attached to pouch 5 according to any method known to those of ordinary skill in the art, such as through the use of adhesives or by a mechanical locking mechanism. Flange 55 and entrance aperture 50 can be provided in any shape or size, such as circular, oblong, heart-shaped, and can be symmetrical or non-symmetrical.

Although illustrated as a coupling-type pouch in the Figures, pouch 5 is not limited and can include any of a wide variety of ostomy pouches known and used in the art. For example, in some embodiments, pouch 5 can include a wafer for adhesive attachment directly to the body or to a body-side coupling member. To this end, one of ordinary skill in the art would appreciate that pouch 5 can take any of a wide variety of shapes other than the embodiments depicted in the Figures herein. That is, the pouch can be provided in any shape or size depending on the intended use thereof, i.e., whether the device is intended for bedridden patients or active patients suffering from incontinence. For example, elongated bags that are principally tubular or rectangular are typically utilized by bedridden patients and elderly incontinence sufferers. For more active patients, the ostomy device can be anatomically shaped such that the device follows the contours of the body and can be worn inconspicuously by the wearer under normal garments. Thus, pouch 5 can be designed to provide sufficient volume for fluid (such as urine) under a variety of wearing conditions. Pouch 5 can be fashioned as a drainable pouch or a collection-only pouch, but is preferably (but not necessarily) a disposable-type ostomy pouch.

II.B. Pouch Materials

As set forth above, pouch 5 can be formed from first and second walls 25, 30 of polymeric material. Polymeric materials, such as thermoplastic polymeric films, can be particularly useful in ostomy pouches due to their liquid handling properties, ease of processability, and low cost. Polymeric films suitable for use with the presently disclosed subject matter include (but are not limited to) films comprising polyethylene, polypropylene, polystyrene, polyvinyl chloride, polyamide, polymethyl methacrylate, low density polyethylene, high density polyethylene, polyurethane, polyepoxide, polyethylene terephthalate, polyethylene terephthalate, polycarbonate, and/or any derivatives or combinations thereof. It should be understood that there is no limit to the materials that can be used to construct walls 25, 30, so long as the pouch constructed therefrom is liquid impermeable.

Generally, first and second walls 25, 30 can be constructed from multilayer or monolayer materials. Typically, however, the films employed will have two or more layers to incorporate a variety of properties, such as, for example, sealability, gas impermeability, and toughness into a single film. Thus, in some embodiments, the films comprise a total of from about 1 to about 20 layers; in some embodiments, from about 4 to about 12 layers; and in some embodiments, from about 5 to about 9 layers. Accordingly, the disclosed films can comprise 1, 2, 3, 4, 5, 6, 7, 8, 9, 10, 11, 12, 13, 14, 15, 16, 17, 18, 19, or 20 layers. One of ordinary skill in the art would also recognize that films suitable for use in first and second walls 25, 30 can comprise more than 20 layers, such as in embodiments comprising microlayering technology.

Walls 25, 30 can be provided in sheet or film form and can be any of the films commonly used for the disclosed type of packaging. Accordingly, walls 25, 30 can comprise one or more barrier layers, seal layers, tie layers, abuse layers, and/or bulk layers. The polymer components used to fabricate walls 25, 30 can also comprise appropriate amounts of other additives normally included in such compositions. For example, slip agents (such as talc), antioxidants, fillers, dyes, pigments, radiation stabilizers, antistatic agents, elastomers, and the like can be added to the disclosed films. See, for example, U.S. Pat. No. 7,205,040 to Peiffer et al.; U.S. Pat. No. 7,160,378 to Eadie et al.; U.S. Pat. No. 7,160,604 to Ginossatis; U.S. Pat. No. 6,472,081 to Tsai et al.; U.S. Pat. No. 6,222,261 to Horn et al.; U.S. Pat. No. 6,221,470 to Ciacca et al.; U.S. Pat. No. 5,591,520 to Miqliorini et al.; and U.S. Pat. No. 5,061,534 to Blemberg et al., the disclosures of which are hereby incorporated by reference in their entireties.

In some embodiments, the polymeric materials use to form walls 25, 30 can comprise at least one layer comprising an absorbent material. Particularly, in some embodiments, the layer comprising the permeable material is positioned adjacent to a permeable layer, such that fluid can be directed to the absorbent layer. The fluid together with the absorbent material in the absorbent layer can form a gel that is contained within the layer of the film.

The polymeric materials used to form walls 25, 30 can be constructed by any suitable process known to those of ordinary skill in the art, including (but not limited to) coextrusion, lamination, extrusion coating, and combinations thereof. See, for example, U.S. Pat. No. 6,769,227 to Mumpower, the content of which is herein incorporated by reference in its entirety.

First and second walls 25, 30 can have any total thickness desired, so long as the film used provides the desired properties for the particular packaging operation in which it is used, e.g., optics, modulus, ductility, quietness, seal strength, and the like. Final web thicknesses can vary, depending on process, end use application, and the like. Typical thicknesses can range from about 0.1 to 20 mils; in some embodiments, about 0.3 to 15 mils; in some embodiments, about 0.5 to 10 mils; in some embodiments, about 1 to 8 mils; in some embodiments, about 1 to 4 mils; and in some embodiments, about 1 to 2 mils. For example, in some embodiments, the thickness can be from about 2 to about 12 mils.

In some embodiments, walls 25, 30 can be transparent (at least in the non-printed regions). The term “transparent” as used herein can refer to the ability of a material to transmit incident light with negligible scattering and little absorption, enabling objects to be seen clearly through the material under typical unaided viewing conditions (i.e., the expected use conditions of the material). The transparency of the walls can be at least about any of the following values: 20%, 25%, 30%, 40%, 50%, 65%, 70%, 75%, 80%, 85%, and 95%, as measured in accordance with ASTM D1746. In some embodiments, walls 25, 30 can comprise at least one color or tint (such as, but not limited to, white, beige, etc.) to impart a desired color to the pouch.

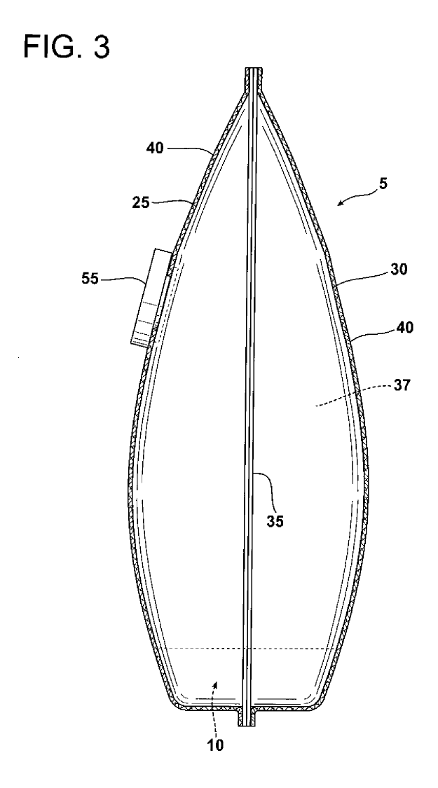

In some embodiments, the outer surfaces of one or both of first and second walls 25, 30 can be provided with a relatively soft, breathable fabric cover 40 to enhance wearer comfort and prevent the outer surfaces of the first and second walls from adhering to the skin or clothing of the user. Cover 40 can be formed from any of a wide variety of materials known in the art, such as (but not limited to) woven materials (such as cotton) and/or nonwoven materials of polyethylene, polypropylene, nylon, or polyester. FIG. 3 illustrates one embodiment of cover 40 applied to the outer surfaces of walls 25, 30. The cover layers can be secured to each other by welding around a peripheral seal, as would be known to those of ordinary skill in the art.

II.C. Sachet 10

As depicted in FIG. 1a, pouch 5 comprises at least one sachet 10 that houses absorbent material 20. Absorbent material 20 can comprise any absorbent material that is capable of absorbing and retaining fluids, such as urine. For example, absorbent material 20 can include (but is not limited to) one or more of the following: airfelt; creped cellulose wadding; meltblown polymers, including coform; chemically stiffened, modified, or cross-linked cellulosic fibers; tissue, including tissue wraps and laminates; absorbent foams; absorbent sponges; superabsorbent polymers; absorbent gelling materials; compressed pulp fiber, or any other known absorbent material or combinations of materials.

Thus, in some embodiments, absorbent material 20 can comprise one or more superabsorbent polymers. Suitable superabsorbent polymers can be selected from natural, synthetic, and modified natural polymers and materials. For instance, the superabsorbent materials can be inorganic materials (such as silica gels) or organic compounds (i.e., crosslinked polymers, such as sodium neutralized polyacrylic acid). In some embodiments, a suitable superabsorbent material can be capable of absorbing at least about 15 times its weight in fluid; in some embodiments, at least about 20 times its weight in fluid; and in some embodiments, at least about 25 times its weight in fluid. The superabsorbent material can be in the form of individual particles, in powder form, or in fiber form.

In some embodiments, one or more additives can be mixed with the absorbent material. Such additives can include (but are not limited to) gel-block preventatives (e.g., carbon particles), odor absorbers (e.g., activated charcoal, zinc, and the like), odor maskers (e.g., fragrances), substances that reduce the rate of bacterial growth (e.g., benzyl alkonium chloride), color masking or neutralizing components, and the like.

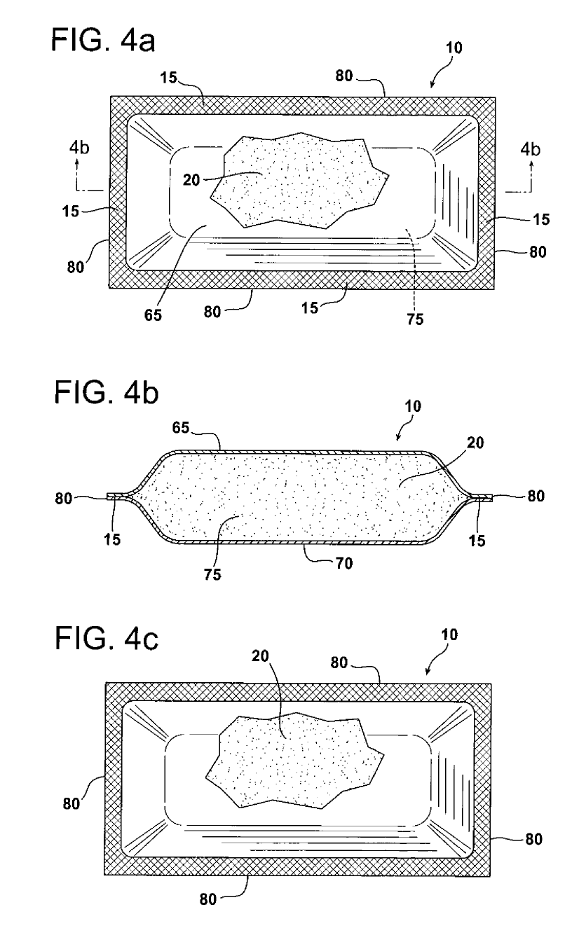

As illustrated in FIGS. 4a and 4b, in some embodiments, the permeable sachet can be constructed from two separate sheets 65, 70 that have been sealed on all edges 80 to define an interior compartment 75 for receiving absorbent material 20. In some embodiments, sachet 10 comprises at least one frangible seal 15 configured on at least one edge 80. As used herein, the term “frangible seal” refers to a seal that is sufficiently durable to allow normal handling of the sachet but will rupture or substantially rupture under desired conditions, such as exposure to a liquid and/or an increase in pressure. Frangible seals are known to those of ordinary skill in the art. See, for example U.S. Pat. No.6,983,839 to Bertram et al. and U.S. Patent Application Publication No. 2006/0093765 to Mueller, the entire disclosures of which are incorporated by reference herein.

In some embodiments, frangible seal 15 of sachet 10 can be constructed using two incompatible films. For example, two incompatible films (such as tissue paper and polyethylene) can be sealed together with an absorbent material positioned in between the two films to form a frangible seal. When exposed to fluid, the absorbent material absorbs the fluid, thereby weakening or breaking the frangible seal. The fluid then contacts the absorbent material housed within the interior of the sachet. The absorbent materials swell, increasing the pressure within the sachet and rupturing the frangible seal and/or at least one sachet film to release the absorbent material into the interior of the ostomy pouch.

In some embodiments, frangible seal 15 can be constructed by introducing an incompatible and absorbent film (such as fabric and/or paper tissue) in between the sealing areas of the frangible seal such that the edges of the absorbent film extend to both sides of the seal. Once fluid enters interior 37 of the ostomy pouch, it flows into sachet 10 through the absorbent film of the frangible seal by means of a wicking action. As a result, the absorbent material housed within the sachet swells. The resultant pressure ruptures at least one frangible seal 15 and/or sachet film 65, 70 and allows the entire absorbent material to come into contact with the remaining fluid in the interior of the pouch.

Alternatively or in addition, frangible seal 15 can be constructed by introducing an incompatible and/or soluble film (such as polyvinyl alcohol ((PVOH)), fibrous material, dissolvable adhesives, or paper, for example) in between the sealing areas of the frangible seal. In use, the soluble film of the frangible seal will dissolve upon contact with fluid, thereby allowing the absorbent materials housed within the sachet to contact and absorb the fluid. The resultant swelling of the absorbent material will rupture the remaining seals of the sachet and allow the entire portion of absorbent material to immobilize the fluid. The sachet material that houses the absorbent material can dissolve, such that there are no solid parts remaining in the pouch contents. Alternatively, in some embodiments, the sachet material includes at least some non-soluble material such that debris can remain in the pouch and is disposed with the pouch contents.

Continuing, in some embodiments, frangible seal 15 can be constructed using a printable, non-sealing ink. Particularly, the ink can be printed in a desired pattern (such as, for example, a zig zag pattern) with open channels. As fluid is deposited into the interior of pouch 5, it contacts the sachet and enters the interior of the sachet by flowing through the open channels. As the fluid is absorbed by absorbent material 20, the swelling ruptures at least one frangible seal 15 and/or sachet film 65, 70, releasing the full amount of absorbent material into the interior of the ostomy pouch.

In some embodiments, frangible seal 15 can be constructed using a water-soluble adhesive with or without the use of an absorbent film. Thus, as fluid enters the interior of the ostomy pouch, the fluid dissolves the adhesive, thereby weakening the frangible seal and allowing the fluid to enter sachet 10. As a result, the absorbent material expands as fluid is absorbed, thereby rupturing at least one frangible seal 15 and/or sachet film 65, 70 and releasing the full amount of absorbent material into the interior of the ostomy pouch.

It should be understood that the presently disclosed subject matter also includes embodiments wherein sachet 10 does not include a frangible seal. In these embodiments, sachet 10 can be constructed from a soluble film (such as, but not limited to, PVOH or paper). As illustrated in FIG. 4c, in these embodiments, the seals around the perimeter of the sachet are hard seals (i.e., non-frangible). As fluid enters the interior of the ostomy pouch and contacts sachet 10, the fluid dissolves the film. Once the sachet film dissolves and fluid is allowed to contact the absorbent material housed within the sachet, the absorbent material expands in size, thereby rupturing the sachet and releasing the full amount of absorbent materials into the interior of the ostomy pouch.

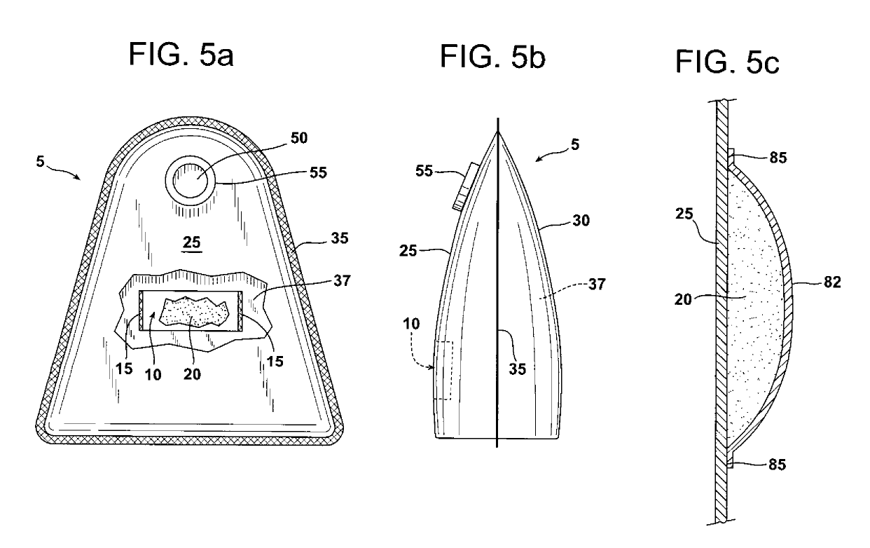

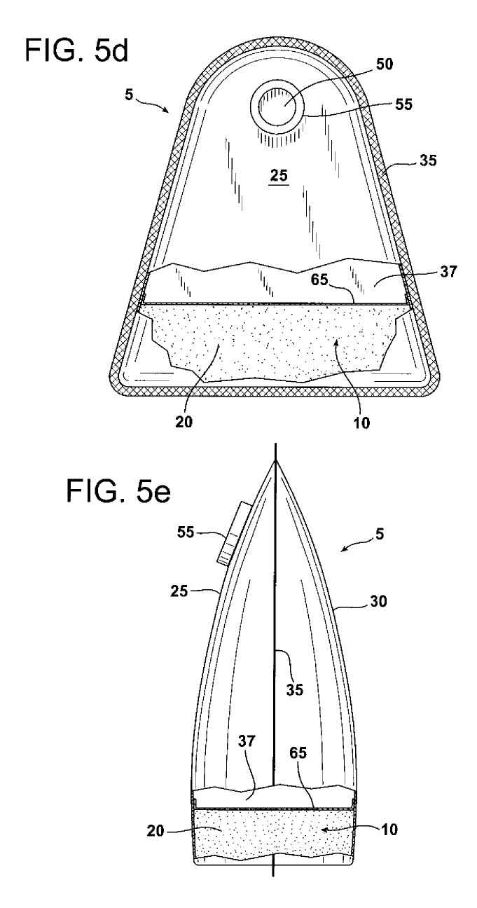

During manufacture of pouch 5, sachet 10 can be included within the interior of the pouch prior to sealing the pouch. To this end, the sachet can freely move about the interior of the pouch, as illustrated in FIG. 1a. Alternatively or in addition, in some embodiments, sachet 10 can be attached to a wall of the interior of the pouch by any of a wide variety of methods, including adhesive or welding, as depicted in FIGS. 5a and 5b. Such embodiments prevent the sachet from moving and also serve to positively locate the sachet during manufacture.

The presently disclosed subject matter also includes embodiments wherein the sachet is positioned within the interior of the pouch and formed between a portion of film and at least one wall of the pouch. Specifically, in some embodiments, the absorbent material can be contained between a wall of the pouch film and a paper tissue, nonwoven, or dissolvable film. FIG. 5c illustrates one such embodiment wherein absorbent material 20 is positioned in the interior of the pouch. Particularly, the sachet is constructed from first wall 25 of the ostomy pouch and a film (which in some embodiments can be paper tissue or dissolvable film) 82 that has been sealed about the perimeter of the absorbent material via seal 85. In some embodiments, seal 85 is a frangible seal. In some embodiments, once fluid contacts film 82, the film dissolves, thereby releasing the absorbent material 20 into the interior of pouch 5. Alternatively or in addition, in some embodiments, once fluid contacts seal 85, the seal weakens, thereby allowing fluid to enter the interior of the sachet. In still further embodiments, film 82 can be fluid permeable such that fluid directly passes into the interior of the sachet to contact the absorbent material housed therein.

FIGS. 5d and 5e illustrate a further embodiment of sachet 10 positioned within the pouch interior and formed between a portion of sachet film and at least one wall of the pouch. Specifically, sachet 10 can be constructed by confining absorbent material 20 in an area of pouch 5 by means of sachet film 65. Sachet film 65 can be adhered to pouch walls 25, 30 using any method known to those of ordinary skill in the art, including heat sealing and/or the use of adhesives. In some embodiments, sachet film 65 can be sealed to pouch films 25, 30 with a frangible seal as set forth herein above. For example, in some embodiments, the frangible seal is adhered to the pouch walls using a dissolvable adhesive or coating such that when fluid contacts the seal, it weakens or ruptures, thereby allowing fluid to enter the interior of the sachet and contact the absorbent materials housed therein. In some embodiments, sachet film 65 can be a soluble film. In these embodiments, as fluid contacts sachet 10, it weakens sachet film 65 until fluid is allowed to pass through the film into the sachet interior 75. In some embodiments, sachet film 65 comprises a fluid permeable film, such that fluid directly passes through the film into the sachet interior. Once fluid passes into the interior of sachet 10, the absorbent materials expand as they absorb the fluid, thereby completely rupturing the frangible seal and/or sachet film 65.

### III. Methods of Using Pouch 5

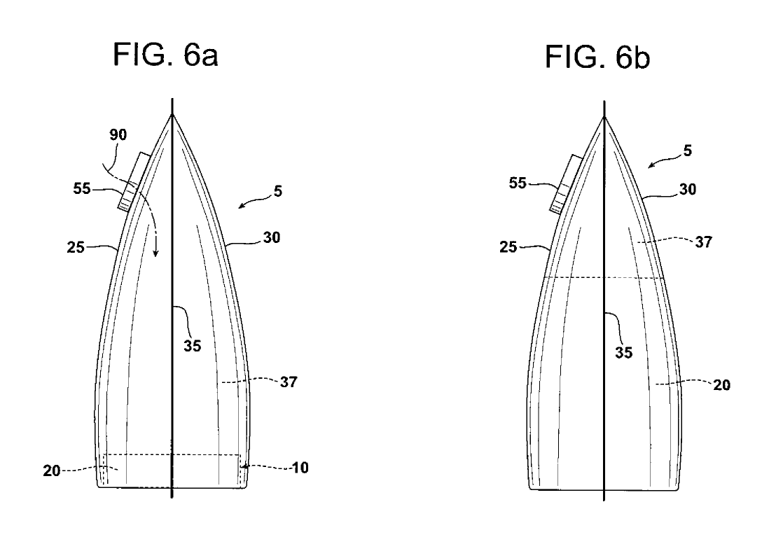

In use, pouch 5 can be connected to a stoma such that wastes can enter the interior compartment of the pouch. Thus, fluid (such as urine and the like) entering pouch 5 follows the path indicated by arrow 90 in FIG. 6a. Particularly, as illustrated, the fluid enters pouch interior 37 through entrance aperture 50 and flows into the pouch interior. Once fluid contacts sachet 10, a sachet frangible seal and/or a sachet soluble film weakens, allowing fluid to enter the sachet interior. As a result, the absorbent material housed within the sachet absorbs the fluid. Alternatively or in addition, in embodiments wherein sachet 10 comprises a fluid-permeable film, fluid passes directly into the interior of the sachet, allowing the absorbent material housed therein to absorb the fluid. Specifically, once the fluid contacts sachet 10, it is able to pass (via frangible seal 15, a porous sachet film, and/or a fluid-permeable sachet film) into the interior of the sachet where it is absorbed by absorbent material 20. Absorbent material 20 can use chemicals or involve chemical processes currently known to those skilled in the art to absorb, solidify, or otherwise capture the fluid. For example, in some embodiments, the absorbent material interacts with the fluid to form a gel, thereby immobilizing the fluid. As more fluid is collected and absorbed by absorbent material 20, the absorbent material will expand and rupture the frangible seal completely and/or rupture the sachet films to be dispersed within the interior of the pouch, as shown in FIG. 6b. Thus, release of the absorbent material from the interior of the sachet is triggered by the rupture of the frangible seal (which is triggered by exposure to fluid).

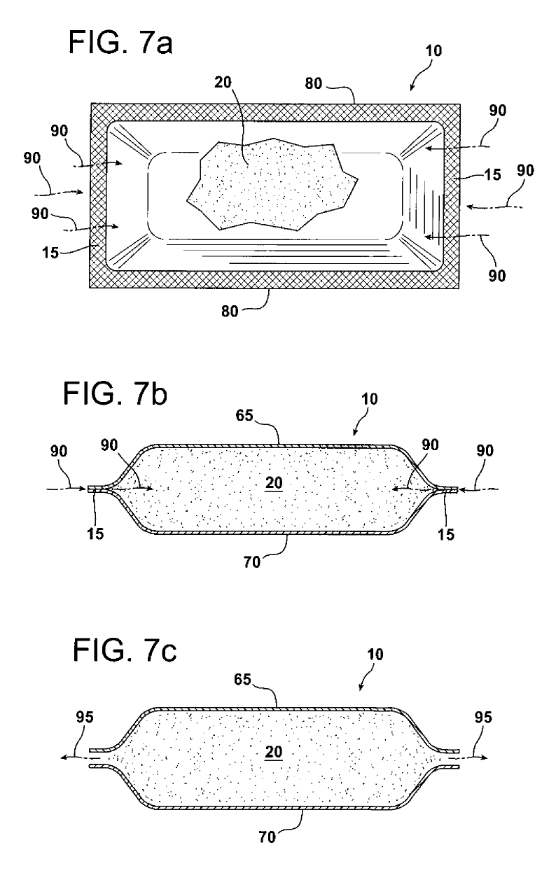

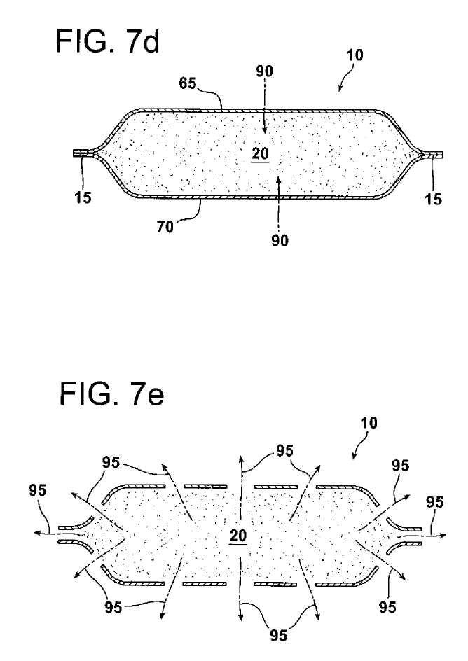

FIGS. 4a-4b , and 7a-7e illustrate the modifications to sachet 10 during use. FIGS. 4a and 4b depict sachet 10 prior to exposure to fluid, with frangible seals 15 still intact. FIGS. 7a and 7b illustrate that in some embodiments, frangible seals 15 can be weakened once they have been exposed to fluid (the action of the fluid is represented by arrows 90). As a result, fluid is allowed to permeate into the interior of the sachet. The absorbent material housed within the interior of the sachet absorbs the liquid and expands, thereby rupturing frangible seal 15, as illustrated in FIG. 7c. Absorbent material 20 is then freed from the sachet (as represented by arrows 95) and is deposited into the interior of the pouch to absorb fluid.

Alternatively or in addition, in some embodiments, at least one of films 65, 70 used to construct sachet 10 is a fluid-permeable film. In these embodiments, once fluid enters pouch 5 and contacts sachet 10, the fluid directly enters the interior of the sachet (represented by arrows 90) to contact the absorbent material housed therein, as illustrated in FIG. 7d. As the absorbent material absorbs the fluid and expands, frangible seal 15 ruptures, freeing the absorbent material from the interior of the sachet, as illustrated in FIG. 7c. Alternatively or in addition, in some embodiments, once the absorbent material absorbs the fluid and expands, sachet films 65, 70 can also rupture to free the absorbent material from the inside of the sachet, as illustrated by FIG. 7e.

It should be appreciated that in some embodiments, at least one of sachet films 65, 70 can comprise a soluble film. In these embodiments, as the absorbent material housed within the sachet interior swells in response to contact with a fluid, the soluble film and/or frangible seal 15 can rupture to free the absorbent material from the sachet (represented by arrows 95), as illustrated in FIG. 7e.

### V. Benefits of the Presently Disclosed Subject Matter

The presently disclosed subject matter provides a pouch for storing wastes that can be easily handled without spillage of its content and that can be easily disposed of after use without the danger of fouling the surrounding environment. To this end, the disclosed pouch beneficially immobilizes waste materials until they can be disposed of.

The disclosed pouch also reliably confines waste residues therein for considerable lengths of time without leakage.

Since the disclosed pouch includes absorbent materials that can gellify fluids that enter the interior of the pouch, there is no sloshing of the fluid inside the pouch during movement. As a result, the pouch is suitable for use as an activity or sports pouch, enabling the user to more easily engage in activities with much less risk of personal embarrassment.

Further, the addition of an odor absorbent (such as, but not limited to, absorbent charcoal) into the sachet or pouch can further enhance the user experience and has been evaluated as an additional benefit compared to the filters of ostomy bags in use today that require gas to pass through and can become blocked and ineffective.

One of ordinary skill in the art would recognize that the benefits described herein above are non-limiting. To this end, the presently disclosed subject matter can include benefits not specified above.

What is claimed is:

1. An ostomy pouch comprising:
a. a first wall and a second wall joined together to define a closed compartment having an interior;
b. an aperture formed in one of the first or second walls of the pouch and in communication with the pouch interior;
c. a sachet positioned within the pouch interior, the sachet comprising:
i. at least one of the following:
1. a frangible seal;
2. a soluble film;
3. a fluid-permeable film; and
ii. at least one absorbent material housed within the interior of the sachet;
wherein the sachet is configured such that the frangible seal, soluble film, or fluid-permeable film allows fluid to contact the absorbent material housed within the interior of the sachet, thereby increasing the pressure within the sachet interior and rupturing the frangible seal, soluble film, fluid-permeable film, or combinations thereof.

2. The pouch of claim 1, wherein the first and second walls are constructed from polymeric materials selected from the group comprising: polyethylene, polypropylene, polystyrene, polyvinyl chloride, polyamide, polymethyl methacrylate, low density polyethylene, high density polyethylene, polyurethane, polyepoxide, polyethylene terephthalate, polyethylene terephthalate, polycarbonate, and combinations thereof.

3. The pouch of claim 1, wherein at least one of the first and second walls comprise at least one layer comprising an absorbent material.

4. The pouch of claim 1, wherein the aperture is of a predetermined size and includes means for fitting the aperture around a stoma.

5. The pouch of claim 1, wherein the absorbent material comprises at least one superabsorbent polymer.

6. The pouch of claim 5, wherein the absorbent material further comprises at least one of the following: gel block preventative, odor absorber, odor masker, bactericide, color masker, and combinations thereof.

7. A method of making an ostomy pouch, the method comprising:
a. forming a pouch comprising a first wall and a second wall joined together to define a closed compartment having an interior;
b. providing an aperture formed in one of the first or second walls of the pouch and in communication with the pouch interior;
c. providing a sachet positioned within the pouch interior, the sachet comprising:
i. at least one of the following:
1. a frangible seal;
2. a soluble film;
3. a fluid-permeable film; and
ii. at least one absorbent material housed within the interior of the sachet;
wherein the sachet is configured such that the frangible seal, soluble film, or fluid-permeable film allows fluid to contact the absorbent material housed within the interior of the sachet, thereby increasing the pressure within the sachet interior and rupturing the frangible seal, soluble film, fluid-permeable film, or combinations thereof.

8. The method of claim 7, wherein the pouch first and second walls are constructed from polymeric materials selected from the group comprising: polyethylene, polypropylene, polystyrene, polyvinyl chloride, polyamide, polymethyl methacrylate, low density polyethylene, high density polyethylene, polyurethane, polyepoxide, polyethylene terephthalate, polyethylene terephthalate, polycarbonate, and combinations thereof.

9. The method of claim 7, wherein at least one of the pouch first and second walls comprise at least one layer comprising an absorbent material.

10. The method of claim 7, wherein the pouch aperture is of a predetermined size and includes means for fitting the aperture around a stoma.

11. The method of claim 7, wherein the absorbent material comprises at least one superabsorbent polymer.

12. The method of claim 11, wherein the absorbent material further comprises at least one of the following: gel block preventative, odor absorber, odor masker, bactericide, color masker, and combinations thereof.

13. A method of using an ostomy pouch, the method comprising:
a. providing a pouch comprising:
i. a first wall and a second wall joined together to define a closed compartment having an interior;
ii. an aperture formed in one of the first or second walls of the pouch and in communication with the pouch interior;
iii. a sachet positioned within the pouch interior, the sachet comprising:
1. at least one of the following:
a. a frangible seal;
b. a soluble film;
c. a fluid-permeable film; and
2. at least one absorbent material housed within the interior of the sachet;
b. attaching the aperture to the stomal area of a user;
wherein the sachet is configured such that the frangible seal, soluble film, or fluid-permeable film allows fluid to contact the absorbent material housed within the interior of the sachet, thereby increasing the pressure within the sachet interior and rupturing the frangible seal, soluble film, fluid-permeable film, or combinations thereof.

14. The method of claim 13, wherein the pouch first and second walls are constructed from polymeric materials selected from the group comprising: polyethylene, polypropylene, polystyrene, polyvinyl chloride, polyamide, polymethyl methacrylate, low density polyethylene, high density polyethylene, polyurethane, polyepoxide, polyethylene terephthalate, polyethylene terephthalate, polycarbonate, and combinations thereof.

15. The method of claim 13, wherein at least one of the pouch first and second walls comprise at least one layer comprising an absorbent material.

16. The method of claim 13, wherein the pouch aperture is of a predetermined size and includes means for fitting the aperture around a stoma.

17. The method of claim 13, wherein the absorbent material comprises at least one superabsorbent polymer.

18. The method of claim 17, wherein the absorbent material further comprises at least one of the following: gel block preventative, odor absorber, odor masker, bactericide, color masker, and combinations thereof.
